(19) United States
(12) Patent Application Publication
(10) Pub. No.: US 2005/0059896 A1
(43) Pub. Date: Mar. 17, 2005

(54) Apparatus for, and method of, determining the condition of a patient's heart

(76) Inventor: Budimir Drakulic, Los Angeles, CA (US)

Correspondence Address: ELLSWORTH R. ROSTON, ESQ.;FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/664,711

(22) Filed: Sep. 17, 2003

(51) Int. Cl. A61B 5/0402

(52) U.S. Cl. 600/509

(57) ABSTRACT

Electrodes disposed in rows and columns in a vest provide signals indicating the patient's heart characteristics. Each electrode is in a column different from the column locating the other electrodes. V₁ and V₂ electrodes are in the same row symmetrically relative to the patient's sternum on opposite sides of the sternum. V₄-V₆ electrodes are in different columns in the same row on the right side of the sternum. V₃ electrode is between the V₂ and V₄ electrodes. The V₁-V₆ electrodes are disposed differently for patients of small, medium and large sizes. The electrodes are attached to the patient with at least a particular pressure. Amplifiers attached to the vest and connected to individual ones of the electrodes amplify heart signals, without any noise production while the patient is ambulatory, thereby providing stable heart signals.

This invention relates to a system for, and method of, indicating the condition of a patient's heart. More particularly, the invention relates to a system for, and method of, measuring the condition of a patient's heart on a more sensitive and reliable basis than in the prior art.

### BACKGROUND OF A PREFERRED EMBODIMENT OF THE INVENTION

Heart diseases present the most serious health problem in human beings today. More people die from heart diseases each year than from any other disease or deficiency. Furthermore, each year millions of people are affected by heart diseases, more than suffer from any other disease or deficiency. Partly because of this, health care is devoting more attention is devoted to the heart than any other organ in the human body.

Measurements are made of the condition of the heart. These measurements are known as electrocardiograms (abbreviated as “ecg” or “ekg”). Electrocardiograms of a patient are obtained by attaching electrodes to the patient at strategic anatomical positions on the patient's body and near the patient's heart and recording the signals produced by the heart at these different positions. The positions of electrodes attached to the patient's body have to be precise since variations in the positioning of the electrodes affect the characteristics of the signals produced at the electrodes. These variations in characteristics may in turn affect any diagnoses provided by the patient's doctor as to the condition of the patient's heart.

A number of electrodes are attached to a patient's body to determine the condition of the patient's heart. The number of the electrodes is often generally sufficiently high so that different problems may arise. One problem is that a mispositioning of one electrode on the patient's body may produce a mispositioning of other electrodes on the patient's body. Often, inaccuracies in the placement of one electrode on the patient's body become aggravated by increased inaccuracies in the placement of other electrodes on the patient's body. Furthermore, electrodes and cables can become mixed so that an electrode intended to be disposed at one position on the patient's body becomes disposed at the position where another electrode is intended to be disposed.

A considerable amount of work has been devoted in perfecting equipment for generating electrocardiograms. In spite of this, an array of problems still exists, some of them somewhat basic. If anything, as medicine and diagnoses increase in sophistication, problems tend to become more subtle instead of becoming simplified. Some of these problems may be indicated as follows:

1. The electrocardiogram measurements made are often not sensitive or reliable. This may affect the diagnoses that are made from these measurements.

2. Measurements are usually made on the patient's body while the patient is stationary. This affects the ability to diagnose heart problems since problems often become apparent only when the patient is ambulatory.

3. Patients are of different sizes. This affects the positioning of the electrodes on the patient and the sensitivity and reliability of the measurements that are made on patients of different sizes.

4. Although there are often a number of electrodes in equipment for providing measurements, the number of electrodes is still quite limited. This often affects the sensitivity and reliability of the measurements that are made. Furthermore, if the number of electrodes becomes increased, the attachment of the electrodes to the proper positions on the patient's skin becomes a long and mentally anguishing process. It often leads to mix-ups between the positioning of the electrodes on the patient's body. When the number of electrodes in a electrocardiogram unit is accordingly limited, the ability of the equipment to detect deficiencies in the functioning of the patient's heart, and to detect among a large range of different deficiencies, becomes accordingly limited.

5. The electrocardiogram equipment is cumbersome. This requires the patient to visit a doctor's office or a hospital to have the electrocardiogram made. The patient has to remain almost completely immobile in the doctor's office or hospital until the electrocardiogram is completed. This “ideal” situation often will not reveal cardiac problems. Furthermore, a patient should be ambulatory and performing everyday activities when an electrocardiogram is taken. A stationary disposition of the patient is not conducive to producing an electrocardiogram with optimal characteristics.

6. The pressure and attachment of the different electrodes to the patient's skin is not uniform and is often not precise and reliable. This causes a variable to be produced in the condition of the signals provided at the different electrodes. This variable prevents the signals produced at the electrodes from truly indicating the condition of the patient's heart at the positions of the electrodes.

7. Cables and amplifiers are attached to the electrodes. The amplifiers affect the signals produced at the electrodes.

Implantable devices and drugs are now available to treat various problems relating to a patient's heart. However, the equipment for determining the condition of the patient's heart is not advancing as rapidly as the implantable devices and drugs for treating the patient's heart when problems in the patient's heart are detected.

### BRIEF DESCRIPTION OF A PREFERRED EMBODIMENT OF THE INVENTION

Electrodes disposed in rows and columns in a vest provide signals indicating the patient's heart condition. Each electrode is in a column different from the columns locating the other electrodes. V₁-V₂ electrodes are in the same row symmetrically relative to the patient's sternum on opposite sides of the sternum. V₄-V₆ electrodes are in different columns in the same row on the right side of the sternum. A V₃ electrode is between the V₂ and V₄ electrodes. The V₁-V₆ electrodes are disposed differently for patients of small, medium and large size. The electrodes are attached to the patient with at least a particular pressure. Amplifiers attached to the vest and connected to individual ones of the electrodes amplify heart signals, without any noise production, while the patient is ambulatory, thereby providing stable heart signals.

The patient's vest and the equipment on the vest address the problems specified in paragraphs 1-7 in the previous section.

### BRIEF DESCRIPTION OF THE DRAWINGS

In the drawings,

FIG. 1 is a schematically elevational view of a skeleton of a patient and shows the disposition on the patient of electrodes for determining the condition of the patient's heart;

FIG. 2 is a schematic elevational view of a vest disposed on a patient and shows an illustrative disposition of electrodes on the vest when the patient has a small size;

FIG. 3 is a schematic elevational view of the vest disposed on the patient and shows an illustrative disposition of electrodes on the vest when the patient has a medium size;

FIG. 4 is a schematic elevational view of the vest disposed on the patient and shows an illustrative disposition of electrodes on the vest when the patient has a large size;

FIG. 4a is a schematic elevational view of the vest disposed on a patient and illustratively shows another disposition of electrodes on the vest when the patient has a large size;

FIG. 5 is a schematic view of a switching arrangement for introducing, from each of the electrodes to an individual one of a plurality of amplifiers, signals indicating the characteristics of the patient's heart at the position where the electrode is attached to the patient's body;

FIGS. 6, 7 and 8 are circuit diagrams of an amplifier which is used to amplify the signals from the electrodes shown in FIGS. 2-4 and 4a;

FIG. 9 shows how the amplifiers shown in FIGS. 6, 7 and 8 are attached to the vest shown in FIGS. 2-4 and 4a;

FIG. 10 is a fragmentary sectional view showing how the electrodes are coupled to the patient's skin;

FIG. 11 is an enlarged schematic diagram of inflatable members for adjusting the pressure applied by each electrode to the skin of the patient so that the pressure has at least a particular value; and

FIG. 12 is a schematic diagram of a vest and electrodes disposed on the vest at positions, other than those shown in FIGS. 2-4 and 4a, to detect heart problems other than those detected by the electrodes shown in FIGS. 2-4 and 4a.

### DETAILED DESCRIPTION OF A PREFERRED EMBODIMENT OF THE INVENTION

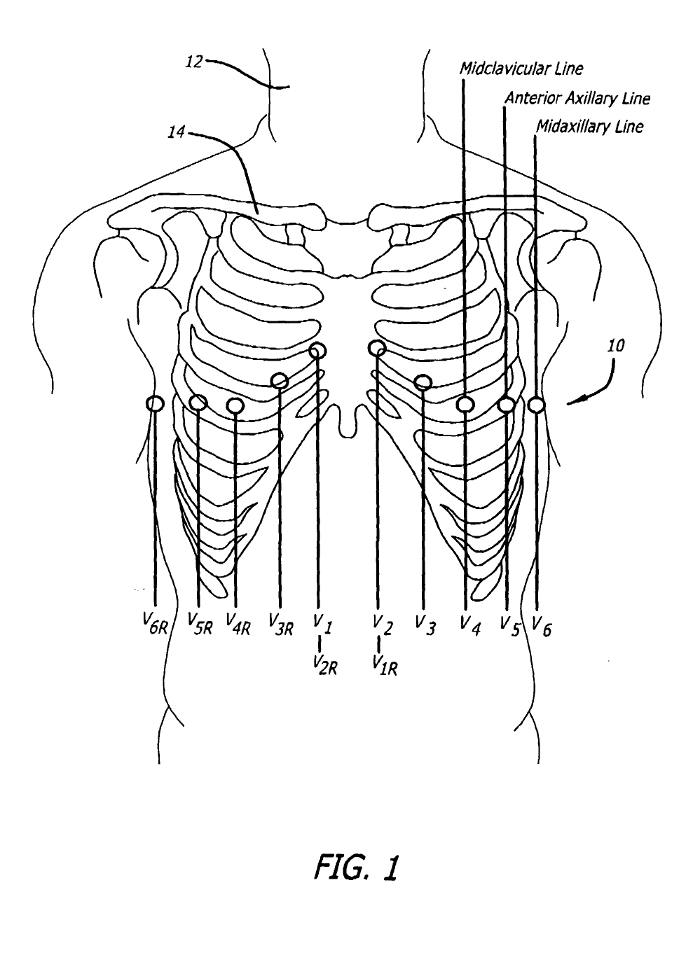

FIG. 1 is a front elevational view showing a skeleton, generally indicated at 10, of a patient 12 and showing the positioning of electrodes attached to the skin of the patient to measure the condition of the patient's heart. FIG. 1 shows a midclavicular line extending vertically. This is a vertical line that extends vertically downwardly from the mid position of the collarbone 14. A midaxillary line is also shown. This line extends downwardly from the patient's armpit. An anterior axillary line is also shown. This line is approximately midway between the midclavicular line and the midaxillary line.

The position of an electrode commonly designated as V₂ in FIG. 1 is also shown. The electrode V₂ is approximately at the fourth intercostal space at the left sternum border. An electrode V₄ is positioned approximately at the mid clavicular line of the fifth intercostal space downwardly from the collarbone at the left side of the patient. An electrode V₃ is positioned midway between the electrodes V₂ and V₄ in a displaced relationship horizontally between the electrodes V₂ and V₄. An electrode V₅ is positioned at approximately the anterior axillary line at approximately the same horizontal level as the electrode V₄. An electrode V6 is positioned at approximately the midaxillary line at approximately the same horizontal level as the electrodes V₄ and V₅. All of the electrodes V₂-V₆ are located on the left side of the patient.

The electrodes may also include an electrode V₁, which has the same position on the right side of the sternum as the electrode V₂ on the left side of the sternum. This causes the electrodes V₁ and V₂ to be symmetrically disposed relative to the sternum. Electrodes V_{3R}, V_{4R}, V_{5R} and V_{6R} may respectfully have approximately the same positions on the right side of the patent 12 as the positions of the electrodes V₃, V₄, V₅ and V₆ on the left side of the patient. The positions for the electrodes V₁, V₂ and V₃-V₆ are well known in the prior art.

The electrodes may also include four limb electrodes, namely,
RA—right arm
LA—left arm
RL—right leg, and
LL—left leg.

The electrode RA provides the signal at the right arm and the electrode LA provides the signal at the left armpit. The electrode RL indicates the characteristics of the signal at the right leg and the electrode LL indicates the characteristics of the signal at the left leg. The electrodes RA, LA, RL and LL are well known in the prior art.

Rather than placing the electrodes RA, LA, RL and LL on the patient's arms and legs, an ambulatory electrocardiogram can be provided by placing these electrodes on the patient's chest such as in a Mason-Likar electrode placement. For example, in a Mason-Likar electrode placement, the RA and LA electrodes are placed on the patient's chest just below respectively the right clavicle and the left clavicle or collarbone. Similarly, in a Mason-Likar electrode placement, the RL and LL electrodes are placed on the right and left sides, respectively, of the lower edge of the patient's rib cage.

It will be appreciated that FIG. 1 and the discussion above relate only to one arrangement of electrodes. If more precision or information are required in determining the characteristics of the patient's heart, additional electrodes may be attached to the patient's body at strategic positions to obtain additional measurements. This will be seen from subsequent discussions. The signals produced at these additional electrodes may be instrumental in diagnosing defects in, or problems with a patient's heart that may not be easily apparent to a specialist who relies only on the signals produced by the electrodes shown in FIG. 1 and described above. It will be appreciated that more than two hundred electrodes can be applied to a patient's torso to measure characteristics of the patient's heart.

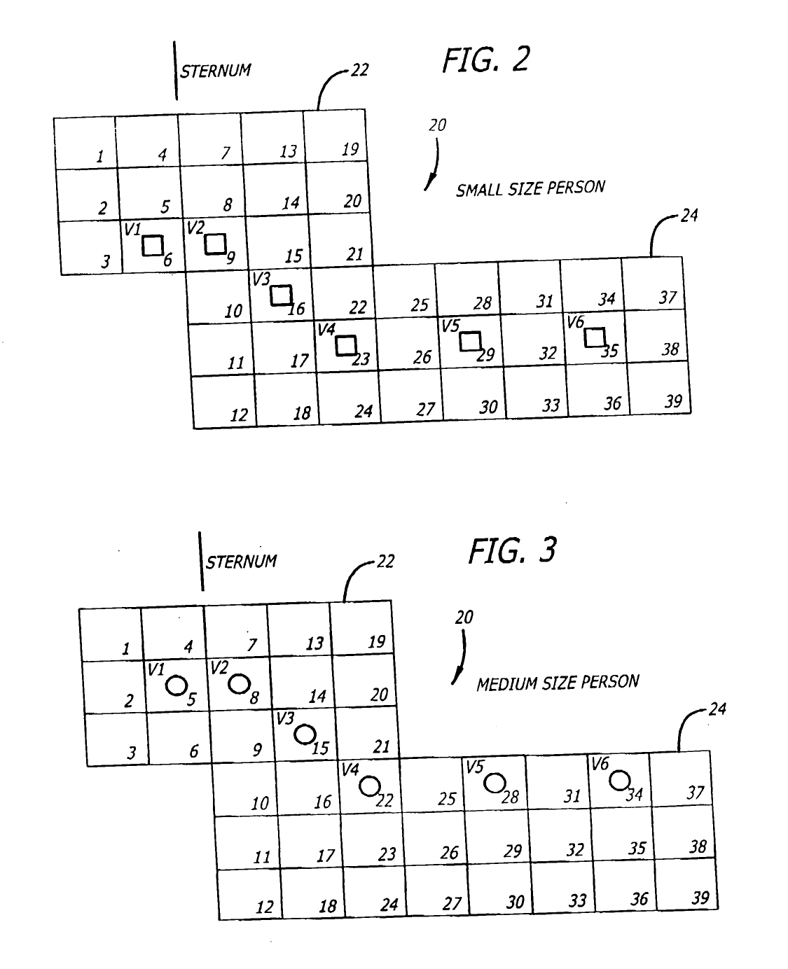

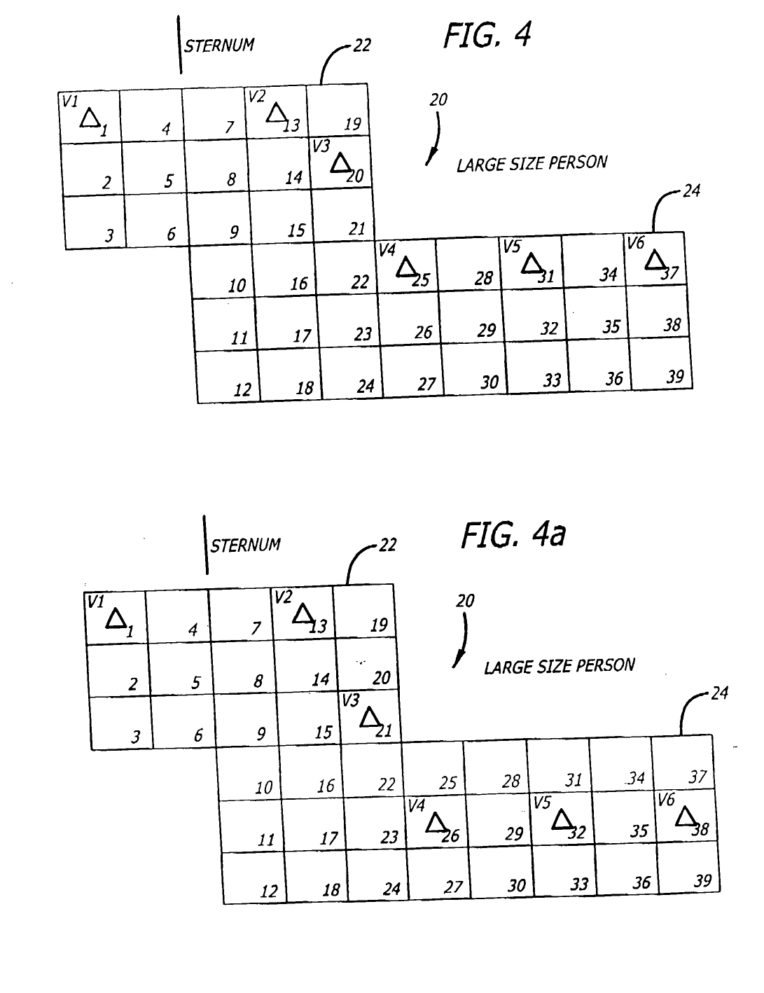

FIGS. 2, 3 and 4 show a vest, generally indicated at 20, which may be included in one embodiment of the invention. FIGS. 2, 3 and 4 show the same vest 20. FIG. 2 shows electrodes V₁-V₆ in the vest 20 when it is used for patients of small size; FIG. 3 shows the electrodes V₁-V₆ in the vest 20 when it is to be used for patients of medium size, and FIG. 4 shows the electrodes V₁-V₆ in the vest 20 when it is to be used for patients of large size.

In each of FIGS. 2, 3 and 4, the vest 20 has a plurality of positions which are disposed in rows and columns. Each position preferably has the same dimensions of length and height as the other positions. In each of FIGS. 2, 3 and 4, each of the positions is provided with characteristics to receive an electrode. In each of FIGS. 2, 3 and 4, there is a first portion 22 with three (3) horizontal rows each having five (5) positions. Each of the rows is in a vertical relationship with the other rows. In this way, there are five (5) columns each having three (3) positions. The portion 22 is provided at upper right positions in the front portion of the vest 20.

In each of FIGS. 2, 3 and 4, the vest 20 has a second portion 24 defined by three (3) horizontal rows each having eight (8) positions. The rows in the portion 24 are disposed in a stacked relationship. The first three (3) positions in each row in the portion 24 are disposed below the last three (3) positions in the rows of the portion 22 so that each of the three (3) columns defined by the overlapping relationship between the portions 22 and 24 has six (6) positions. In each of FIGS. 2, 3 and 4, the vest 20 is disposed on the patient so that the sternum of the patient is at the line common to the second (2^{nd}) and third (3^{rd}) columns in the portion 22.

FIG. 2 illustrates a typical disposition of the electrodes V₁-V₆ when the patient has a small size. In FIG. 2, the disposition of the electrodes V₁-V₆ is indicated by small squares each identified by an individual one of the electrodes V₁-V₆. As will be seen, the electrodes V1 and V₂ are symmetrically disposed relative to the sternum and are in the same horizontal row on opposite sides of the sternum. The electrodes V₄, V₅ and V₆ are in the same horizontal row on the same side of the sternum as the electrode V₂. The electrode V₃ is between the electrodes V₂ and V₄ and is in a different column and row than the electrodes V₂ and V₄. Each of the electrodes V₁-V₆ is in a separate column different from the columns in which the other ones of the electrodes V₁-V₆ are located.

FIG. 3 illustrates a disposition of the electrodes V₁-V₆ on the vest 20 when the patient has a medium size. The disposition of the electrodes V₁-V₆ in FIG. 3 is indicated by circles each identified by an individual one of the electrodes V₁-V₆. As will be seen, the electrodes V₁ and V₂ are on opposite sides of the sternum in the same horizontal row and are symmetrically disposed relative to the sternum. The electrodes V₄, V₅ and V₆ are in different columns in the same horizontal row and the electrode V₃ is between the electrodes V₂ and V₄ and is in a different column and row than the electrodes V₂ and V₄. Each of the electrodes V₁-V₆ is in a different column than the other ones of the electrodes V₁-V₆.

FIG. 4 illustrates a disposition of the electrodes V₁-V₆ on the vest 20 when the patient has a large size. The disposition of the electrodes V₁-V₆ in FIG. 4 is indicated by triangles each identified by an individual one of the electrodes V₁-V₆. As will be seen, the electrodes V₁-V₂ are on opposite sides of the sternum in the same horizontal row and are symmetrically disposed relative to the sternum. The electrodes V₄, V₅ and V₆ are in different columns in the same horizontal row and the electrode V₃ is between the electrodes V₂ and V₄ and in a different column and row than the electrodes V₂ and V₄. Each of the electrodes V₁-V₆ is in a different column than the other ones of the electrodes V₁-V₆.

The disposition of the electrodes V₁-V₆ in the vest 20 in FIGS. 2, 3 and 4 is only illustrative. Different positionings of the electrodes V₁-V₆ in the vest 20 than those shown in FIGS. 2-4 may be provided without departing from the scope of the invention. For example, an alternative disposition of the electrodes V₁-V₆ in the vest 20 for patients of a large size is shown in FIG. 4a. In FIG. 4a, the electrodes V₁-V₆ are again shown as triangles. These triangles are respectively designated as V₁-V₆ The electrodes V₁ and V₂ are disposed in the same horizontal row on the opposite sides of the sternum and in equally spaced relationship to the sternum. The electrodes V₄, V₅ and V₆ are disposed in different columns in the same row. The electrode V₃ is equally spaced from the electrodes V₂ and V₄ and is in a column and row different from the columns and rows in which the electrodes V₂ and V₄ are located.

It will also be appreciated that the portions 22 and 24 do not have to have the configurations shown in FIGS. 2, 3, 4 and 4a. There are fifteen (15) positions in the portion 22 and twenty-four (24) positions in the portion 24. The portions 22 and 24 do not have to have these respective numbers of positions. Furthermore, the number of portions can be more than two (2) without departing from the scope of the inventions.

When a patient first visits the patient's doctor to have a physical checkup, the doctor or the doctor's nurse may take an electrocardiogram. For example, the doctor or the doctor's nurse may provide a disposition of the electrodes V₁-V₆ as shown in FIG. 2 and as described above when the patient has a small size. The doctor or the doctor's nurse should make a record of the disposition of the electrodes as shown in FIG. 2. Every time thereafter that the doctor or the doctor's nurse takes an electrocardiogram of the patient, the doctor or the doctor's nurse should provide a disposition of the electrodes V₁-V₆ as shown in FIG. 2. The purpose of this is to provide for a comparison between the signals produced at each of the electrodes V₁-V₆ for the patient at progressive instants of time. In this way, the patient's doctor is able to determine if there is any change in characteristics, or in the condition, of the patient's heart and, if so, what the change of characteristics or condition is.

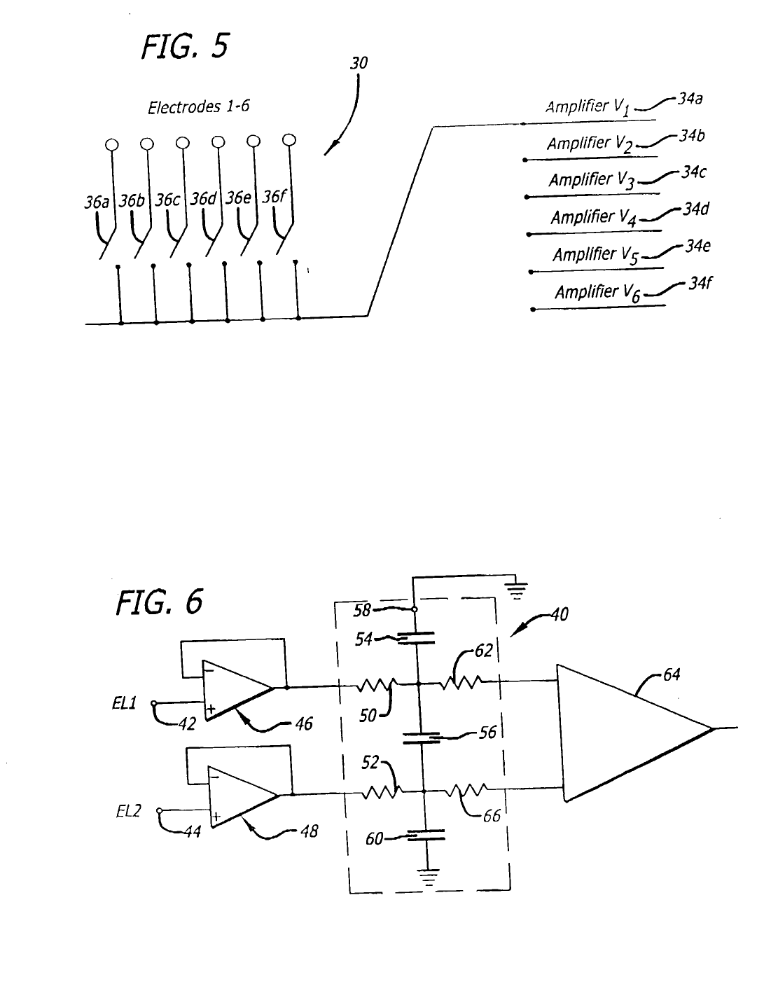

FIG. 5 shows a switch mechanism, generally indicated at 30, for introducing the signals from the electrodes V₃ to an amplifier 34c respectively associated with the electrode V₃. The switch mechanism 30 in FIG. 5 may be associated with the electrode V₃ to introduce the signals from the electrode V₃ to the amplifier 34c. The amplifier 34d may constitute one of a plurality of amplifiers 34a-34f each of which receives the signals from an individual one of the electrodes V₁-V₆ through an individual one of the switches 36a-36f.

Six switches 36a-36f are illustratively shown in FIG. 5 for the amplifier 34c, one for each of the six (6) positions in the column which includes the electrode V₃. Counting from the top downwardly in FIG. 5, the electrode V₃ is disposed in the fourth (4^{th}) position when a small person is providing an electrocardiogram as shown in FIG. 2. This causes the switch 36d to close and the signals on the electrode V₃ to pass through the switch to the amplifier 34c for amplification. It is believed that a person of ordinary skill in the art will be able to extrapolate from the discussion above to provide signals for introduction to each of the amplifiers 34a-34f in accordance with the signals produced on the individual one of the electrodes V₁-V₆ associated with the amplifier.

The construction of each of the amplifiers 34a-34f corresponds to the construction of the other ones of the amplifiers 34a-34f. The construction of each of the amplifiers 34a-34f is disclosed and claimed in application Ser. No. 10/611,696 filed on Jul. 1, 2003 in the USPTO for AMPLIFIER SYSTEM FOR DETERMINING PARAMETERS OF A PATENT in the name of Budimir Drakulic as a sole inventor and assigned of record to the assignee of record of this application. If the Examiner should have any questions relating to the construction and operation of the amplifier 34a-34f, the Examiner should refer to co-pending application Ser. No. 10/611,696.

FIGS. 6, 7 and 8 in this application respectively correspond to FIGS. 1, 2 and 6 in co-pending application Ser. No. 10/611,696. FIG. 6 is a circuit diagram, primarily in block form, of an amplifier system, generally indicated at 40, constituting a preferred embodiment of the invention. The amplifier system 40 includes a pair of electrodes 42 and 44 each of which is suitably attached to skin at a selective position on the patient's body. The electrodes 42 and 44 preferably have an identical construction. The electrode 42 is positioned at a selective position on the skin of the patient's body to produce signals related to the functioning characteristics of an organ in the patient's body. The organ may illustratively be the patient's heart, brain or the patient's stomach or intestines. The electrode 44 is positioned on the skin of the patient's body at a position displaced from the selective position to provide reference signals. The difference between the signals at the electrodes 42 and 44 represents the functioning characteristics of the selected one of the patient's organs.

The signals on the electrode 42 are introduced to an input terminal of an amplifier generally indicated at 46. The amplifier 46 also has a second input terminal which is connected to the output of the amplifier. In this way, the amplifier acts as a unity gain. The amplifier 46 may be purchased as an OPA 129 amplifier from the Burr-Brown Company which is located in Phoenix, Ariz. In like manner, the signals from the electrode 44 are introduced to an input terminal of an amplifier, generally indicated at 48, which may be identical to the amplifier 46. The amplifier 48 has an input terminal which is connected to the output terminal of the amplifier to have the amplifier act as a unity gain.

Resistors 50 and 52 respectively extend from the output terminals of the amplifiers 46 and 48. The resistor 50 is connected to first terminals of capacitors 54 and 56. The second terminal of the capacitor 54 receives a reference potential such as ground. A connection is made from the resistor 52 to the second terminal of the capacitor 56 and to a first terminal of a capacitor 60, the second terminal of which is provided with the reference potential such as ground. The resistors 50 and 52 may have equal values and the capacitors 54 and 60 may also have equal values.

One terminal of a resistor 62 is connected to the terminal common to the capacitors 54 and 56. The other terminal of the resistor 62 has a common connection with a first input terminal of an amplifier 64. In like manner, a resistor 66 having a value equal to that of the resistor 62 is connected at one end to the terminal common to the capacitors 56 and 60 and at the other end to a second input terminal of the amplifier 64.

Since the amplifiers 46 and 48 have identical constructions, they operate to provide signals which represent the difference between the signals on the electrodes 42 and 44. This indicates the functioning of the patient's organ which is being determined by the amplifier system 60. Although the electrodes 42 and 44 are displaced from each other on the skin of the patient's body, they tend to receive the same noise signals. As a result, the difference between the signals on the output terminals of the amplifiers 46 and 48 do not include any noise.

The electrodes 42 and 44 can respectively provide an impedance as high as approximately 10⁶ ohms to the amplifiers 46 and 48. Each of the amplifiers 46 and 48 respectively provides an input impedance of approximately 10¹⁵ ohms. This impedance is so large that it may be considered to approach infinity. This causes each of the amplifiers 46 and 48 to operate as if it has an open circuit at its input. The output impedance of each of the amplifiers 46 and 48 is approximately 50 ohms to 75 ohms.

Because of the effective open circuit at the input of each of the amplifiers 46 and 48, the output signal from each of the amplifiers 46 and 48 corresponds to the input signal to the amplifiers and does not have any less magnitude compared to the amplitude of the input signal to the amplifier. This is important in view of the production of signals in the microvolt or millivolt region in the electrodes 42 and 44.

The capacitors 54, 56 and 60 and the resistors 50 and 52 provide a low-pass filter and a differential circuit and operate to eliminate the noise on the electrodes 42 and 44. The capacitors 54, 56 and 60 also operate to provide signals which eliminate the commonality between the signals in the electrodes 42 and 44 so that only the signals individual to the functionality being determined relative to the selected organ in the patient's body remain. The capacitors 54, 56 and 60 operate as a low pass filter and pass signals in a range to approximately one kilohertz (1 KHz). The signals having a frequency above approximately one kilohertz (1 KHz) are attenuated.

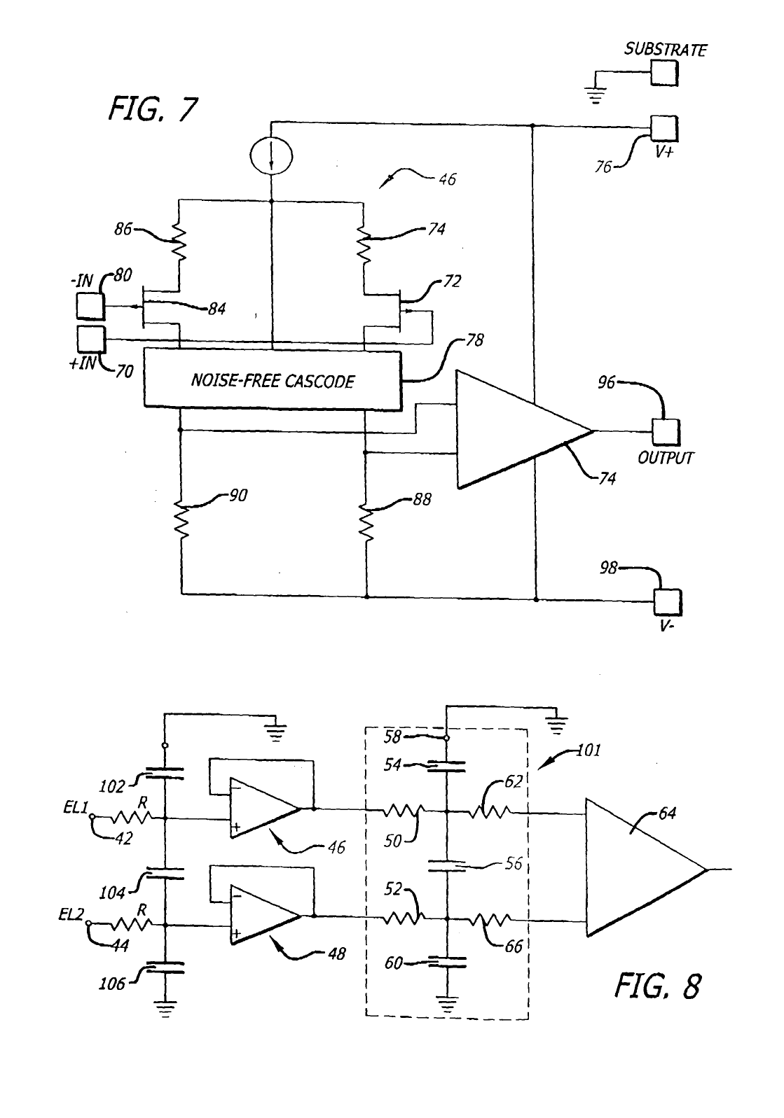

The amplifiers 46 and 48 are identical. Because of this, a description of the construction and operation of the amplifier 46 will apply equally as well to the amplifier 48. The amplifier 46 is shown in detail in FIG. 7. It is manufactured and sold by Burr-Brown in Phoenix, Ariz. and is designated by Burr-Brown as the OPA 129 amplifier.

As shown in FIG. 7, the amplifier 46 includes an input terminal 70 which receives the signals at the electrode 42 and introduces these signals to the gate of a transistor 72. The source of the transmitter 72 receives a positive voltage from a terminal 76 through a resistor 74. The emitter of the transistor 72 is common with an input terminal in a noise free cascode 78.

Another terminal 80 receives the signals on the electrode 44 and introduces these signals to a gate of a transistor 84. A connection is made from the source of the transistor 84 to one terminal of a resistor 86, the other terminal of which receives the voltage from the terminal 76. The emitter of the transistor 84 is common with an input terminal in the noise-free cascode 78. The resistor 86 has a value equal to that of the resistor 74 and the transistors 72 and 84 have identical characteristics.

First terminals of resistors 88 and 90 having equal values are respectively connected to output terminals in the noise-free cascode 78 and input terminals of an amplifier 94. The amplifier 94 provides an output at a terminal 96. The output from the terminal 96 is introduced to the input terminal 80. The amplifier 94 receives the positive voltage on the terminal 76 and a negative voltage on a terminal 98. Connections are made to the terminal 98 from the second terminals of the resistors 88 and 90.

The transistors 72 and 84 operate on a differential basis to provide an input impedance of approximately 10¹⁵ ohms between the gates of the transistors. The output impedance from the amplifier 46 in FIG. 6 is approximately fifty (50) ohms to seventy-five (75) ohms. Because of the high input impedance of approximately 10¹⁵ ohms, the amplifier 46 provides an input impedance approaching infinity. This causes the amplifier 46 to provide the equivalent of an open circuit at its input. This causes substantially all of the voltage applied to the input terminal 70 to be provided at the output of the amplifier 46. This is facilitated by the low impedance of approximately fifty ohms (50 ohms) to seventy-five (75) ohms at the output of the amplifier 46. This voltage has characteristics corresponding to the characteristics of the voltage at the electrode 42.

The output signals from the amplifiers 46 and 48 are respectively introduced to the terminal common to the capacitors 54 and 56 and to the terminal common to the capacitors 56 and 60. The capacitors 54, 56 and 60 operate as a low-pass filter to remove noise and to provide an output signal representing the difference between the signals on the electrodes 42 and 44.

The capacitors 54, 56 and 60 correspond to the capacitors C2, C1 and C3 in a low pass filter 76 in application Ser. No. 10/293,105 (attorney's file RECOM-61860) filed on Nov. 13, 2002 in the USPTO and assigned of record to the assignee of record in this application. The capacitors C2, C1 and C3 in application Ser. No. 10/293,105 are included in the low pass filter 96 in FIG. 8-1 (also shown in FIG. 4) of such application. The low pass filter 96 eliminates noise and passes signals through a frequency range to approximately one kilohertz (1 KHz). If any further information may be needed concerning the construction and operation of the low pass filter, reference may be made to co-pending application Ser. No. 10/293,105 to obtain this information.

FIG. 8 shows a preferred embodiment, generally indicated at 101, constituting a modification of the amplifier system 40 shown in FIG. 6. It is identical to the amplifier system 40 shown in FIG. 6 except that it includes capacitors 102, 104 and 106 respectively corresponding to the capacitors 54, 56 and 60 also shown in FIG. 6. The capacitors 102, 104 and 106 are connected as a low pass filter at the inputs of the amplifiers 46 and 48. Like the capacitors 54, 56 and 60, the capacitors 102, 104 and 106 operate as a low pass filter. The addition of the capacitors 102, 104 and 106 provides an additional low-pass filter. Furthermore, it assures that the amplifier system 101 provides stable output signals even when the amplifier system is included in an ambulatory system for measuring the heart characteristics of a patient.

The amplifier shown in FIGS. 6, 7 and 8 described above has certain important advantages. It provides an amplification of the signals introduced to the amplifier without the passage of any high frequency noise through the amplifier. It provides an amplification of the signals introduced to the amplifier without any change in the phase or amplitude characteristics of the signal. The faithful amplification of the signals without any high frequency noise and without any changes in the phase and amplitude characteristics of the signal are beneficial in the amplifier in providing signals which faithfully correspond to the characteristics of the signals produced at the electrodes. This facilitates a reliable analysis by the patient indicator of the patient's electrocardiogram and an accurate determination of what defects or deficiencies, if any, exist in the patient's heart.

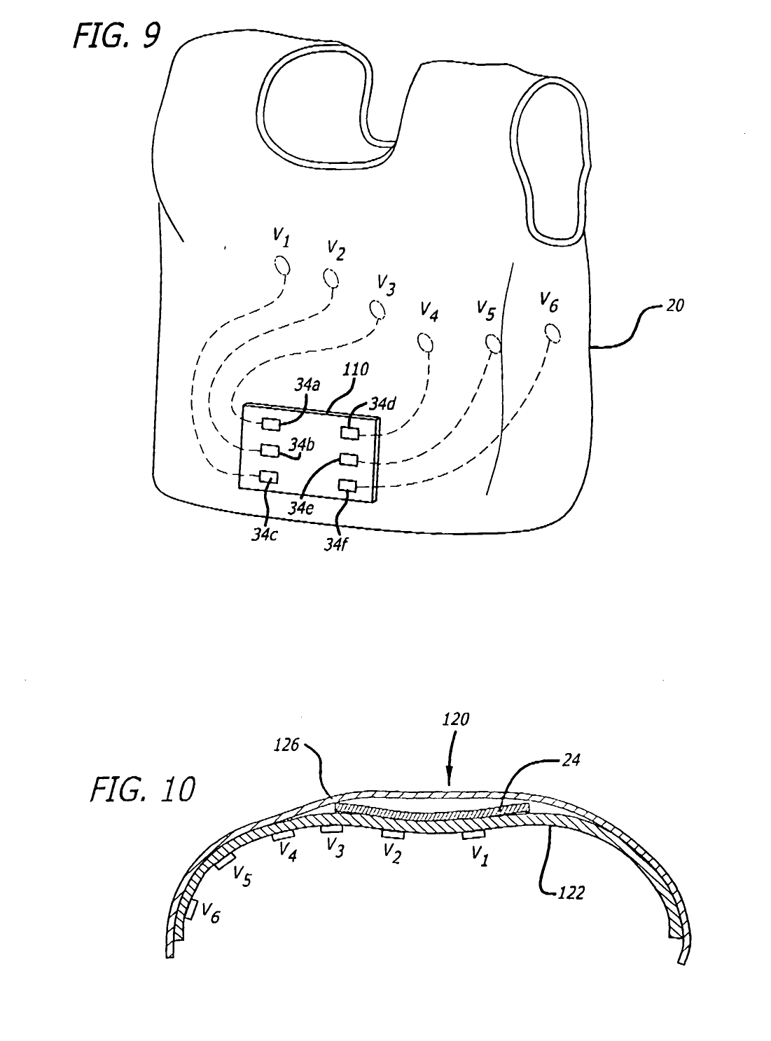

FIG. 9 shows how the amplifiers 34a-34f are attached to the vest 20. A carrying plate 110 is integrated into the vest 20 and the amplifier 34a-34f are attached to the carrying plate 110. The carrying plate 110 may be made from a thin, lightweight and strong material to provide minimal inconvenience to the patients and to facilitate the amplifications of the signals from the electrodes such as the electrode V₁-V₆, when the patient is wearing the vest.

FIG. 10 shows structure, generally indicated at 120, for attaching an electrode (e.g., one of the V₁-V₆ electrodes) to the vest 20 and for supporting the vest 20 on the patient's body. The structure 120 includes an electrode positioning substrate 122 which supports the electrodes V₁-V₆ An anatomically contoured (rigid) brace 124 is disposed across the entire vertical area of the patient. A flexible tensioning strap 126 envelops the substrate 122 and the brace 124 and retains the substrate and the brace fixedly against the patient's body. It will be appreciated that the structure 120 constitutes only one way of holding the electrodes V₁-V₆ against the patient's body.

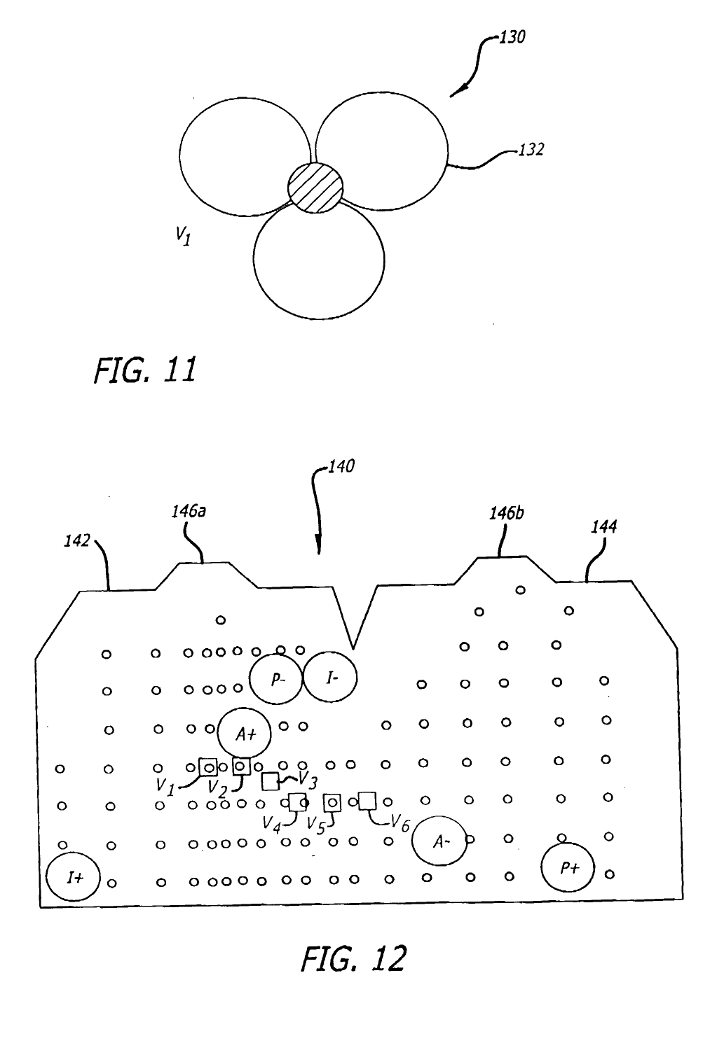

FIG. 11 schematically illustrates an arrangement, generally indicated at 130, for providing at least a particular pressure of an electrode (e.g. V₁-V₆) against a patient's body. The electrode (e.g. V₁) may be disposed at the center of inflatable bulbs or bubbles 132. The bubbles 132 are inflatable to hold the electrode V₁ against the skin of the patient. The inflation can be continued until the pressure of the electrode against the patient's skin is at least a particular value. This pressure assures that the electrode will produce a signal indicative of the characteristics of the patient's heart at the position of the electrode.

FIG. 12 shows a vest, generally indicated at 140, which wraps completely around a patient. The front of the vest 140 is indicated at 142 and the rear of the vest is indicated at 144. The head of a patient is indicated at 146a and 146b. Electrodes V₁-V₆ are indicated by squares and by the respective markings of V₁-V₆. Designations P− and P+ indicate positions where signals are produced to indicate whether a patient has posterior myocardial infarction. Designations A+ and A− indicate positions where signals are produced to indicate whether a patient has anterior myocardial infarction. Designations I+ and I− indicate positions where signals are produced to indicate whether a patient has inferior myocardial infarction. As will be seen, the designations P+, P−, A+, A−, I+ and I− indicate electrode positions. These indicate that measurements are at least occasionally made in positions outside of the portions 22 and 24.

Although this invention has been disclosed and illustrated with reference to particular embodiments, the principles involved are susceptible for use in numerous other embodiments which will be apparent to persons of ordinary skill in the art. The invention is, therefore, to be limited only as indicated by the scope of the appended claims.

1. In combination for providing signals at different positions in a patient's heart,
a vest constructed to be worn by the patient,
a plurality of electrodes,
a plurality of positions in the vest for receiving the electrodes, individual positions in the plurality being disposed to receive the electrodes for measuring the characteristics of the patient's heart when the patient has a small, medium or large size, thereby to obtain signals indicating the characteristics of the patient's heart when the patient has a small, medium or large size, and
amplifiers responsive to the signals on the electrodes at the individual positions in the vest for providing signals indicating the characteristics of the patient's heart at the different positions for the patient when the patient has a small, medium or large size.

2. In a combination as set forth in claim 1 wherein
the electrodes are positioned at the individual positions in the vest to measure V₁-V₆ positions in the heart for the patient when the patient has a small, medium or large size and wherein
the amplifiers provide the signals at the individual ones of the V₁-V₆ positions of the patient's heart when the patient has the small, medium or large size.

3. In a combination as set forth in claim 1 wherein
the electrodes measure individual ones of the V₁-V₆ positions in the patient's heart when the patient has the small, medium or large size, and wherein
the amplifiers provide signals indicating the characteristics of the patient's heart at the individual ones of the V₁-V₆ positions for the patient when the patient has the small, medium or large size.

4. In a combination as set forth in claim 1 wherein
the positions in the vest are disposed in rows and columns and wherein
each of the amplifiers provides indications of the heart in the individual ones of the rows at an individual one of the columns when the patient has a small, medium or large size.

5. In a combination as set forth in claim 1 wherein
the electrodes at the individual positions are provided with pressures against the body of the patient of at least a particular value to facilitate the production by the patient of signals indicating the characteristics of the patient's heart at the individual positions when the patient has the small, medium or large size.

6. In a combination as set forth in claim 2 wherein
the electrodes measure individual ones of the V₁-V₆ positions in the heart for the patient when the patient has the small, medium or large size and wherein
the amplifiers provide signals indicating the characteristics of the heart at individual ones of the V₁-V₆ positions for the patients when the patient has the small, medium or large size and wherein
the positions in the vest are disposed in rows and columns and wherein
each of the amplifiers provides indications of the heart in individual ones of the rows at an individual one of the columns when the patient has a small, medium or large size and wherein
the electrodes at the individual positions are provided with pressures against the body of the patient of at least a particular value to facilitate the production by the patient of signals indicating the characteristics of the patient's heart at the individual positions.

7. In combination for providing signal indications at individual positions in a patient's heart,
a vest constructed to be disposed on a patient's body for ambulatory movement of the patient while measurements are being made of the characteristics of the patient's heart,
a plurality of electrodes each providing a measurement at an individual one of the positions in the patient of the characteristics of the patient's heart at that position,
the positions on the vest constituting pluralities each plurality being provided to receive an individual one of the electrodes regardless of the size of the patient, and
a plurality of amplifiers, each connected to an individual one of the electrodes to receive signals from an individual one of the electrodes regardless of the size of the patient.

8. In a combination as set forth in claim 7 including
an electrode assembly disposed on the vest and including a plurality of electrodes each connected to provide the signals to an individual one of the amplifiers.

9. In a combination as set forth in claim 7 wherein
the vest is provided with rows and columns of positions and wherein
the electrodes are disposed in individual ones of the positions depending upon whether the patient has a small, medium or large size and wherein
each amplifier is connected to an individual one of the electrodes, when the patient has a small, medium or large size, to provide signals representing an individual position in the patient's heart regardless of the size of the patient.

10. In a combination as set forth in claim 7 wherein
the positions on the vest are disposed in rows and columns and wherein
each amplifier provides signals in an individual one of the columns when the patient has a small, medium or large size and wherein
each amplifier provides an indication of the signal on an individual one of V₁-V₆ positions in the patient when the patient has a small, medium or large size.

11. In a combination as set forth in claim 7 wherein
the electrodes for the patient, when the patient has a small, medium or large size, size are positioned on the vest so that at most only one electrode is disposed in each column of the positions on the vest regardless of the size of the patient.

12. In a combination as set forth in claim 7 wherein
each electrode is disposed on the vest with a pressure against the patient at least equal to a particular value.

13. In a combination as set forth in claim 8 wherein
the electrodes are disposed in rows and columns and wherein
each amplifier is connected to an individual one of the electrodes, when the patient has a small, medium or large size, to provide signals representing individual positions in the patient's heart regardless of the size of the patient and wherein
each amplifier provides signals in an individual one of the columns when the patient has a small, medium or large size and wherein
each amplifier provides an indication of an individual one of V₁-V₆ positions in the patient and wherein
the electrodes for the patient, when the patient has a small, medium or large size, are positioned on the vest so that at most only one electrode is disposed in each column of the positions on the vest regardless of the patient's size and wherein
each electrode is disposed on the vest against the patient with at least a particular pressure.

14. In combination for providing signals at different positions in a patient's heart,
a vest having a plurality of positions for determining the characteristics of the patient's heart,
a plurality of electrodes constructed to be connected to the vest at individual ones of the different positions, and
an inflatable member for inflating the vest with the vest disposed on the patient to press the electrodes against the patient's body for enhancing the passage of the signals from the patient's heart to the electrodes.

15. In a combination as set forth in claim 14,
a member carried by the patient and having a plurality of terminals for receiving the signals from the different electrodes.

16. In a combination as set forth in claim 14 wherein
amplifiers are disposed on the member and are connected to the electrodes and wherein
the member is attached to the vest to provide for the production of signals from the patient's heart at the electrodes, while the patient is ambulatory, without affecting the characteristics of the signals from the patient's heart and wherein
the amplifiers are constructed to amplify the signals from the electrodes without affecting the characteristics of the signals even while the patient is ambulatory.

17. In a combination as set forth in claim 14 wherein
the vest has a plurality of positions, dependent upon the size of the patient, for receiving electrodes to measure the characteristics of the heart at positions V₁-V₆ in the patient's heart.

18. In a combination as set forth in claim 15 wherein
the member is attached to the vest to provide for the production of signals at the electrodes, while the patient is ambulatory, without affecting the characteristics of the signals produced at the different electrodes and wherein
the vest has a plurality of positions, dependent upon the size of the patient, for receiving electrodes to measure the characteristics of the heart at positions V₁ -V₆ on the patient's heart.

19. In a combination for providing signals at different positions in a patient's heart,
a vest constructed to be worn by the patient,
a plurality of positions on the vest, the positions being disposed in rows and columns,
electrodes disposed in the vest at particular positions in the vest, the particular positions being dependent upon the size of the patient wearing the vest,
there being at most only one electrode in each column in the vest regardless of the size of the patient.

20. In a combination as set forth in claim 19,
a plurality of amplifiers each responsive to the signals from the electrode in an individual one of the columns, different from the other columns in which the electrodes are disposed for receiving the signals in the heart.

21. In a combination as set forth in claim 20 wherein
the amplifiers are attached to the vest in a closely coupled relationship to the vest to be carried by the patient in an ambulatory relationship of the patient.

22. In a combination as set forth in claim 20, wherein
the amplifiers are constructed, and are connected to the electrodes, to provide signals of stable characteristics from the electrodes regardless of the size of the patient and even while the patient is ambulatory.

23. In a combination as set forth in claim 20 wherein
the signals from the amplifiers are independent of any noise that may result from the ambulatory nature of the patient.

24. In a combination as set forth in claim 22 wherein
the signals from the amplifiers are independent of any noise that may result from the ambulatory nature of the patient.

25. In a combination as set forth in claim 19,
an inflator for inflating the vest against the patient's body for providing an adjustable pressure of the electrodes against the patient's body.

26. In a combination as set forth in claim 19,
an inflator for inflating the vest against the patient's body to provide at least a particular pressure of each electrode against the patient's body.

27. In a combination as set forth in claim 19 wherein
each of the electrodes is individually inflated against the patient's body to provide at least a particular pressure between the electrode and the patient's body.

28. In a combination as set forth in claim 23 wherein
each of the electrodes is individually inflated against the patient's body to provide, between the electrode and the patient's body, a pressure which is at least a particular value.

29. In a combination as set forth in claim 23
a plurality of amplifiers each responsive to the signals from an individual one of the electrodes in an individual one of the columns, regardless of the patient's size, for amplifying the signals in the electrode.

30. In a combination as set forth in claim 24
a plurality of amplifiers each responsive to the signals from an individual one of the electrodes in an individual one of the columns, regardless of the patient's size, for amplifying the signals in the electrode.

31. In a combination as set forth in claim 20,
each of the electrodes is individually inflated against the patient's body to provide, between the electrode and the patient's body a pressure which at least equals a particular value.

32. In a combination for providing signals at different positions in a patient's heart,
a vest constructed to be worn by the patient,
a plurality of positions disposed on the vest in rows and columns in an upper left portion of the vest and in positions in a lower right portion of the vest, and
electrodes disposed on the vest in positions in the upper right portion of the vest and in positions in the lower left portion of the vest.

33. In a combination as set forth in claim 32 wherein
V₁ and V₂ electrodes are disposed in the upper right portion of the vest regardless of the size of the patient and wherein
the V₁ and V₂ electrodes in the upper right portion of the vest are symmetrically disposed relative to the patient's sternum and wherein
electrodes are disposed in positions in the lower right portion of the vest regardless of the size of the patient.

34. In a combination as set forth in claim 32 wherein
V₄, V₅ and V₆ electrodes are disposed in positions in an individual one of the rows in the lower left portion of the vest regardless of the size of the patient.

35. In a combination as set forth in claim 33 wherein
a V₃ electrode is disposed in either the upper right portion or the lower left portion dependent upon the size of the patient.

36. In a combination as set forth in claim 35 wherein
V₁ and V₂ electrodes are in positions in the upper right portion of the vest in the same horizontal row on opposite sides of the sternum in a symmetrical relationship with the sternum regardless of the size of the patient and wherein
V₄, V₅ and V₆ electrodes are in positions on the lower left portion in the same horizontal row.

37. In a combination as set forth in claim 32 wherein
there are two (2) electrodes in the positions in the upper right portion of the rectangle and two of these electrodes are in the same row in a symmetrical relationship with the sternum of the patient regardless of the size of the patient and wherein
a third electrode is in either the upper right portion of the vest or the lower left portion of the vest in a row and column different from the row and columns of the first two electrodes, the positioning of the third electrode being dependent upon the size of the patient and wherein
there are three electrodes in position in the lower left portion of the vest and all of these are in the same horizontal row but in a row different from the rows locating the other electrodes and in columns different from the other electrodes and from one another, the positioning of the three electrodes being dependent upon the size of the patient.

38. In a combination as set forth in claim 32 wherein
positions are provided in the upper right portion of the rectangle and in the lower left portion of the rectangle to provide for the disposition of electrodes in particular ones of the positions for patients of small, medium and large sizes and wherein
each of the electrodes is in a different column from the other columns regardless of the size of the patient.

39. In a combination as set forth in claim 38 wherein
the upper right portion overlaps the lower left portion and wherein
at least two (2) electrodes are disposed in positions in the upper right portion regardless of the size of the patient and wherein
three electrodes are disposed in the positions in the lower left portion regardless of the size of the patient and wherein
a sixth electrode is disposed in either the upper right portion of the vest or the lower left portion of the vest dependent upon the size of the patient.

40. In combination for providing signals at different positions in a patient's heart,
a vest constructed to be worn by the patient,
a first plurality of positions in the upper left portion of the vest and a second plurality of positions in the lower right portion of the vest,
the positions in the upper left portion and the lower right portion of the vest defining rows and columns, and
electrodes disposed in particular ones of the positions in the upper left portion and the lower right portion of the vest for providing signals indicative of the characteristics of the heart regardless of the size of the patient.

41. In a combination as set forth in claim 40,
the electrodes constituting first electrodes and being disposed relative to the patient's heart and being operative to produce signals indicative of V₁-V₆ positions in the heart regardless of the size of the patient.

42. In a combination as set forth in claim 40,
a third plurality of positions in the lower right portion of the vest and a fourth plurality of positions in the upper left portion of the vest, and
the electrodes constituting first electrodes, and
second electrodes disposed in particular ones of the third and fourth positions for providing signals indicative of the characteristics of the heart at these positions.

43. In a combination as set forth in claim 40,
the second electrodes being disposed relative to the patient's heart and being operative to produce signals indicative of relatively rare heart problems in comparison to the heart problems indicated by the signals from the first electrodes.

44. In combination for providing signals at different positions in a patient's heart,
a vest constructed to be worn by the patient,
a plurality of positions in the vest,
a plurality of electrodes each disposed in an individual one of the plurality of positions and operative to provide signals indicative of the characteristics of the heart at this position, and
a plurality of amplifiers each connected to an individual one of the electrodes to amplify the signals from the individual one of the electrodes, the amplifiers being supported by the vest to facilitate ambulatory movement of the patient with the vest.

45. In a combination as set forth in claim 44,
the amplifiers being constructed to amplify the signals, without producing noise in the signals, during ambulatory movements of the patient with the vest disposed on the patient.

46. In a combination as set forth in claim 44 wherein
electrodes disposed in first positions in the vest provide signals indicative of relatively common problems in the patient's heart and electrodes disposed in second positions in the vest provide signals indicative of relatively rare problems in the heart.

47. In a combination as set forth in claim 46 wherein
the amplifiers are constructed to provide signals indicative of the characteristics of the heart at the positions of the electrodes without any noise resulting from any ambulatory movements of the patient.

48. In a combination as set forth in claim 44 wherein
electrodes disposed at first positions in the vest provide signals indicative of relatively common problems in the patient's heart and electrodes disposed at second positions in the vest provide signals indicative of relatively rare problems in the heart and wherein
the amplifiers are constructed to provide signals indicative of the characteristics of the heart at the positions of the electrodes without any noise resulting from any ambulatory movements of the patient.

49. In combination for providing signals at different positions in a patient's heart,
a vest constructed to be worn by the patient,
a plurality of positions in the vest,
a plurality of electrodes each disposed in an individual one of the positions and operative to provide signals indicative of the characteristics of the heart at this position, and
a plurality of amplifiers each connected to an individual one of the electrodes to amplify the signals from the individual one of the electrodes, the amplifiers being constructed to provide signals indicative of the characteristics of the heart at the positions of the electrodes without any noise resulting from any ambulatory movements of the patient.

50. In a combination as set forth in claim 49,
the electrodes being disposed in the vest at individual positions in the vest for patients of small, medium and large size.

51. In a combination as set forth in claim 49,
each of the electrodes being disposed in a coupled relationship with the patient's skin to apply pressure against the patient's skin with a value greater than a particular value.

52. In a combination as set forth in claim 50,
each of the electrodes being disposed in a coupled relationship to the patient's skin to apply a pressure to the patient's skin at a value greater than a particular value for producing signals indicative of the patient's heart at the position of the electrode.

53. In a combination as set forth in claim 49,
the electrodes being disposed in the vest at individual positions in the vest for patients of small, medium and large sizes to obtain signals indicative of the characteristics of the heart of the patient of small, medium and large sizes.

54. In a combination as set forth in claim 50,
each of the electrodes being disposed in a coupled relationship with the patient's skin to apply a pressure against the patient's skin of at least a particular value,
the electrodes being disposed in the vest at individual positions in the vest for patients of small, medium and large sizes to obtain signals indicative of the characteristics of the hearts of the patients of small, medium and large sizes.

55. In a method of providing signals at different positions in a patient's heart, the steps of:
providing a vest to be worn by the patient,
providing a plurality of positions in the vest for the reception of electrodes,
disposing the electrodes in the vest at individual ones of the positions dependent upon the size of the patient, and
applying the electrodes to the patient's skin at the individual ones of the positions to produce on the electrodes signals indicative of the characteristics of the heart at the positions of the electrodes.

56. In a method as set forth in claim 55, the step of:
adjusting the pressure applied by each electrode against the skin to maintain a pressure of the different electrodes against the skin of at least a particular value.

57. In a method as set forth in claim 55, the step of:
introducing the signals on the electrodes to amplifiers disposed on the vest and having characteristics of amplifying the signals, without the production of noise, while the patient is ambulatory.

58. In a method as set forth in claim 55, the step of:
providing the different positions on the housing in rows and columns and providing for the indication of V₁-V₆ electrodes in columns wherein each of the electrodes is in a column different from the columns in which the other ones of the V₁-V₆ electrodes are located.

59. In a method as set forth in claim 58 wherein
each of the amplifiers provides signals indicative of the patient's heart at the position of an individual one of the V₁-V₆ electrodes by receiving indications from the individual one of the columns in which the individual one of the V₁-V₆ electrodes is located.

60. In a method as set forth in claim 55 wherein
the electrodes include V₁-V₆ electrodes and the V₁-V₆ electrodes occupy individual positions on the vest for each of the patients whether the patients have a small, medium or large size.

61. In a method as set forth in claim 58 wherein
the sizes of the patients constitute small, medium and large and the positions of the V₁-V₆ electrodes for patients having one of the small, medium and large sizes are different from the positions of the corresponding electrodes for patients having other ones of the small, medium and large sizes.

62. In a method as set forth in claim 57, the steps of:
providing the different positions on the housing in rows and column and providing for the indication of V₁-V₆ electrodes in columns wherein each of the electrodes is in a column different from the columns in which the other ones of the V₁-V₆ electrodes are located and wherein
each of the amplifiers provides signals indicative of the patient's heart at the position of an individual one of the V₁-V₆ electrodes by receiving indications from the individual one of the columns in which the individual one of the V₁-V₆ electrodes is located and wherein
the V₁-V₆ electrodes occupy individual positions on the vest for each of the different sizes of the patients.

63. In a method as set forth in claim 57, including the steps of:
the sizes of the patients constitute small, medium and large and the positions of the V₁-V₆ electrodes for the patients having any one of the small, medium and large sizes are different from the positions of the corresponding electrodes for the patients having the other ones of the small, medium and large sizes and wherein
the different positions on the housing are provided in rows and columns and wherein the indications of the V₁-V₆ electrodes are provided in columns such that any one of the electrodes is in a column different from the columns in which the other ones of the V₁-V₆ electrodes are located and wherein
each of the amplifiers provides signals indicative of the patient's heart at the position of an individual one of the V₁-V₆ electrodes by receiving indications from the individual one of the columns in which the individual one of the V₁-V₆ electrodes is located.

64. In a method of providing signals at different positions in a patient's heart, the steps of:
providing a vest to be worn by the patient whether the patient has a small, medium or large size,
providing a plurality of positions in the vest,
disposing V₁-V₆ electrodes in the vest at individual ones of the positions dependent upon whether the patient has a small, medium or large size, and
applying the V₁-V₆ electrodes to the patient's skin at the individual ones of the positions of the electrodes to produce on the electrodes signals indicative of the characteristics of the heart at the positions of the V₁-V₆ electrodes.

65. In a method as set forth in claim 64 wherein
the V₁-V₂ electrodes are disposed on opposite sides of the patient's sternum in a substantially symmetrical relationship with the sternum.

66. In a method as set forth in claim 64 wherein
regardless whether the patient has a small, medium or large size, the V₄, V₅ and V₆ electrodes are disposed on a substantially horizontal line at positions below the V₁-V₂ electrodes and wherein the V₃ electrode is displaced vertically between the V₁-V₂ electrodes and the V₄, V₅ and V₆ electrodes and is displaced horizontally from the V₁ and V₂ electrodes and the V₄, V₅ and V₆ electrodes.

67. In a method as set forth in claim 64 wherein
the positions are disposed in rows and columns and wherein
a plurality of positions are provided in each column and wherein
each of the V₁-V₆ electrodes is disposed, regardless of whether the patient has a small, medium or large size, in a column different from the columns in which the other ones of the V₁-V₆ electrodes are disposed.

68. In a method as set forth in claim 64 wherein
a plurality of amplifiers are provided and wherein
each of the amplifiers is connected to an individual one of the V₁-V₆ whether the patient has a small, medium or large size.

69. In a method as set forth in claim 68 wherein
each of the amplifiers is provided with characteristics of amplifying the signals from the individual one of the electrodes, without the production of noise, while the patient is ambulatory.

70. In a method as set forth in claim 64 wherein
the V₄, V₅ and V₆ electrodes are disposed on a substantially horizontal line at positions below the V₁-V₂ electrodes and the V₃ electrode is disposed vertically between the V₁-V₂ electrodes and the V₄, V₅ and V₆ electrodes and wherein
the positions are disposed in rows and columns and wherein
a plurality of positions are provided in each column and wherein
regardless of whether the patient has a small, medium or large size, each of the V₁-V₆ electrodes is disposed in a column different from the columns in which the other ones of the V₁-V₆ electrodes are disposed and wherein
a plurality of amplifiers are provided and wherein
each of the amplifiers is connected to receive the outputs from the electrode in any of the positions in an individual one of the columns whether the patient has a small, medium or large size and wherein
each of the amplifiers is provided with characteristics of amplifying the signals from the electrode in the individual one of the columns, without the production of noise, while the patient is ambulatory.

71. In a method of providing signals at different positions in a patient's heart, the steps of:
providing a vest to be worn by the patient,
providing a plurality of positions in the vest,
disposing electrodes at individual ones of the positions in the vest,
applying the electrodes to the patient's skin at the individual ones of the positions to produce at the electrodes signals indicative of the characteristics of the patient's heart at the positions of the V₁-V₆ electrodes,
providing a plurality of amplifiers each having characteristics of amplifying the patient's heart signals at an individual one of the electrodes, without any production of noise while the patient is ambulatory, thereby providing stable signals from the patient's heart, and
connecting each of the amplifiers to the individual one of the electrodes.

72. In a method as set forth in claim 71 wherein
the positions on the vest are disposed in rows and columns and wherein
the electrodes are disposed at positions in the rows and columns such that each one of the electrodes is in an individual one of the columns different from the columns in which the other ones of the electrodes are disposed.

73. In a method as set forth in claim 71 wherein
the electrodes constitute V₁-V₆ electrodes and wherein
the V₁-V₂ electrodes are disposed in the same row on opposite sides of the patient's sternum and wherein
the V₄, V₅ and V₆ electrodes are disposed in different columns in the same row and wherein
the V₃ electrode is disposed in a different row and column than the rows and columns in which the other ones of the V₁-V₆ electrodes are disposed.

74. In a method as set forth in claim 71 wherein
the V₁-V₂ electrodes are disposed on the opposite sides of the sternum in a substantially symmetrical relationship with the sternum.

75. In a method as set forth in claim 71 wherein
each of the amplifiers is connected to receive the signals from one of the electrodes in the different positions in an individual one of the columns.

76. In a method as set forth in claim 71 wherein
each of the electrodes is disposed at individual positions dependent upon the size of the patient.

77. In a method as set forth in claim 71 wherein
the electrodes constitute V₁-V₆ electrodes and wherein
the V₁-V₂ electrodes are disposed in the same row on opposite sides of the patient's sternum and wherein
the V₄, V₅ and V₆ electrodes are disposed in different columns in the same row and wherein
the V₃ electrode is disposed in a different row and column than the rows and columns in which the other ones of the V₁-V₆ electrodes are disposed and wherein
the V₁-V₂ electrodes are disposed in a substantially symmetrical relationship with the plaintiff's sternum on the opposite sides of the sternum and wherein
each of the amplifiers is connected to receive the signals from one of the electrodes in the different positions in an individual one of the columns and wherein
each of the electrodes is disposed at individual positions dependent upon the size of the patient.
